(19) United States
(12) Patent Application Publication
(10) Pub. No.: US 2026/0158232 A1
(43) Pub. Date: Jun. 11, 2026

(54) EXHALATION VALVE

(71) Applicant: Loewenstein Medical Technology S.A., Luxembourg (LU)

(72) Inventor: Helmut HECHLER, Herrieden (DE)

(21) Appl. No.: 18/707,258

(22) PCT Filed: Nov. 9, 2021
(86) PCT No.: PCT/EP2021/081151

§ 371 Date: May 3, 2024

(51) Int. Cl. A61M 16/20

(52) U.S. Cl. CPC A61M 16/205; A61M 16/208

(57) ABSTRACT

An exhalation valve comprises a main body comprising a hollow passage with an inlet for receiving respiratory gas from a ventilator and an outlet for supplying respiratory gas to a patient, a branch tube arranged between inlet and outlet and having a free end, an annular channel externally delimited by a wall, surrounding the branch tube and having a free end, at least one outlet opening for respiratory gas exhaled, returned via the outlet and branched off via the branch tube, a cap with a port for attaching a compressed air control line, and a diaphragm arranged at the free ends of the branch tube and the wall, making it possible, for exhaled respiratory gas to pass into the annular channel. The outlet opening which is variable in the outlet direction of the respiratory gas is provided on the annular channel in the region of the branch tube.

The present invention relates to an exhalation valve, which is used as a control valve for the ventilation of patients.

### TECHNOLOGICAL BACKGROUND

Exhalation valves of the type of interest in the present case are used to receive breathing gas from a ventilator and supply it to a patient. The exhalation valve further ensures that the breathing gas exhaled by the patient can be discharged to the surroundings. The valve function of the exhalation valve therefore controls two processes, namely inhalation and exhalation. The control is performed by applying compressed air to a diaphragm when the patient inhales, while compressed air is not applied when the patient exhales. In the latter case, the air exhaled by the patient can lift the diaphragm and escape through the outlet opening. Via an attached pressure measuring line, the pressure in the exhalation valve can be detected and, if necessary, used for evaluation by the ventilator.

The exhalation valve is conventionally located very close to the patient, for example in the immediate vicinity of a breathing mask.

### PUBLISHED PRIOR ART

TW 201143828 A1 discloses an exhalation valve of the generic type, in which the opening for the exhaled breathing gas returned from the patient is located on the opposite side of the exhalation valve from the diaphragm. The outlet opening is connected here to a connector with reduced diameter in order to equilibrate a pressure difference between the inner and outer sides of a valve plate and to reduce noise.

DE 10 2008 026 321 A1 discloses a patient valve with a base body constructed in two parts, the first part of the base body being fitted onto the second part. A third part is furthermore provided, which constitutes the cap and can be connected to the first part of the base body using a bayonet coupling. The outlet opening for the breathing gas coming from the patient is located in the region of the branch tube.

EP 1 512 426 A1 discloses an exhalation valve in which the outlet opening for breathing gas coming from the patient is formed by an opening formed by the base body together with the cap. The opening is rigidly defined in its position on the exhalation valve.

Lastly, EP 3 360 595 B1 discloses an exhalation valve in which the base body in the region of the branch tube and the cap are connected together by means of a bayonet coupling. An outlet port formed laterally on the base body is provided as the outlet opening.

### PROBLEM UNDERLYING THE PRESENT INVENTION

The object of the present invention is to provide an improved exhalation valve.

### SOLUTION OF THE PROBLEM

The aforementioned object is achieved by the features of claim 1. Expedient embodiments of the invention are claimed in the dependent claims.

Owing to the fact that the outlet opening is provided in the wall externally delimiting the annular channel and the outlet opening is variable in respect of the outflow direction of the breathing gas out of the outlet opening, or the annular channel, relative to the base body, or the longitudinal axis of the latter, the flow direction of the breathing gas from the patient leaving the exhalation valve can be varied according to requirements without changing the position of the exhalation valve on the patient. If, for example, the air flow due to the exhaled gas would be, for example, directed counter to the upper body of the patient because of the individual alignment of the exhalation valve relative to the patient, the air flow may be steered in a different direction by adjusting the outflow direction of the outlet opening. Since the outlet opening in the annular channel is located in the region of the branch tube, preferably laterally with respect to the branch tube, the exhaled gas can immediately leave the exhalation valve after emerging from the branch tube without having to flow through constrictions on the exhalation valve, or its base body, which cause turbulences and pressure buildups. In this way, the flow of the exhaled gas inside the exhalation valve can be kept as laminar as possible, which is advantageous.

For varying the outlet opening, a cover is expediently provided, which is variable in its position along the circumference of the annular channel, preferably displaceable with respect to, or along, the wall externally delimiting the annular channel. By rotating, or displacing, the cover relative to the wall externally delimiting the annular channel, the outlet direction of the outlet opening may therefore straightforwardly be varied according to individual requirement.

At the same time, the cap may straightforwardly be connected to the base body by a clamping or snap-fit connection. The snap-fit connection may be formed by an inwardly directed projection on the cap and a complementary latching recess in the wall (or vice versa).

According to a further expedient embodiment, the cap has a recess for receiving the outer part of a valve diaphragm.

For this purpose, the cover may have at least one projection, preferably a multiplicity of projections, which engages or engage in the outlet opening. The outlet opening thereby fulfills a twofold function.

It is advantageous for the cover to be provided as a rotary ring. A rotary ring may straightforwardly be produced as plastic part by the injection-molding method, and also allows particularly simple mounting.

The cover is preferably a cover that has only a part of the full angular extent of a circle, preferably an angular extent in the range of from 180° to 270°. A targeted outflow direction may therefore be adjusted. In addition, the cover may straightforwardly be used when assembling the exhalation valve, by first being stretched apart and then snap-fitted onto the wall.

Owing to the fact that the outlet opening comprises a plurality of elongate holes distributed along the circumference of the annular channel, on the one hand the base body may straightforwardly be produced by the injection-molding method, and on the other hand the elongate holes also offer advantages in respect of improved outflow of the breathing gas out of the valve.

In particular, two neighboring elongate holes may in each case respectively be separated from one another along the circumference of the annular channel only by a web. The web may have concavely configured web boundaries on both sides.

The cover may advantageously be located outside the wall. In this case, this provides very simple mounting with simultaneously good operability.

The annular channel, or the wall externally delimiting the latter, expediently extends at least substantially only onto the region of the branch tube of the base body, and not beyond onto the opposite side of the exhalation valve from the diaphragm. This avoids constrictions and turbulences for the breathing gas coming from the patient.

In order to control the exhalation valve, a diaphragm is provided, which closes the open end of the branch tube by compressed air being applied or, when the compressed air is no longer applied and when the patient exhales, the exhaled breathing gas lifts the diaphragm because of the pressure of the exhaled gas, and can enter the annular channel through the open end of the branch tube and leave the exhalation valve from there through the outlet opening.

Preferably, the diaphragm is fixed by lying on the open end, or free end, of the wall externally delimiting the annular channel and being fixed, preferably firmly clamped, by the cap. In this way, both the base body and the cap may be configured simply in terms of design. At the same time, it ensures simple mounting.

Preferably, the cap may have a recess for fixing the diaphragm, in which the outer edge region of the diaphragm engages.

It is further advantageous if the cap of the diaphragm additionally provides the necessary free space in the direction of the control pressure port, so that the diaphragm can be lifted by the exhaled air pressure and the exhaled gas can escape without significant additional breathing effort.

By virtue of the design according to the invention, the base body may advantageously be produced as a one-piece component.

### DESCRIPTION OF THE INVENTION WITH THE AID OF EXEMPLARY EMBODIMENTS

An expedient embodiment of the exhalation valve according to the invention will be explained in more detail below with the aid of figures of the drawing, in which:

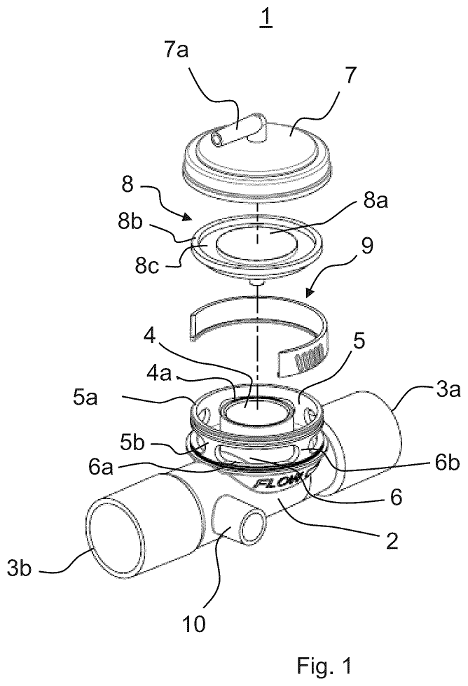

FIG. 1 shows an exploded representation of one example of an exhalation valve according to the invention;

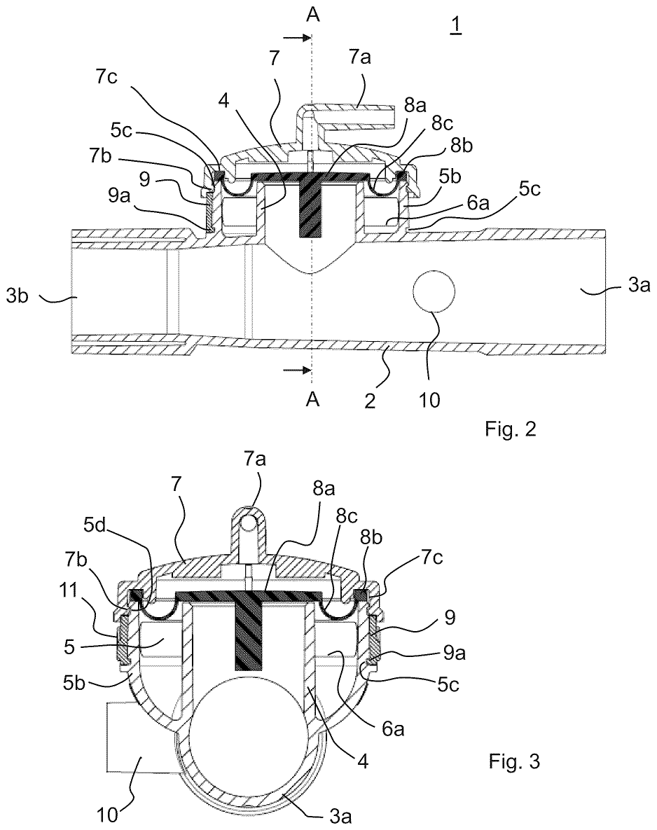

FIG. 2 shows a longitudinal sectional representation through the exhalation valve of FIG. 1; and

FIG. 3 shows a sectional representation along the section plane A-A of FIG. 2.

FIG. 1 depicts an example of an exhalation valve 1 according to the invention as an exploded representation. The exhalation valve 1 comprises a base body 2, which is configured in one piece as a plastic part, preferably produced by the injection-molding method. The base body 2 comprises an inlet 3a, which is intended to receive breathing gas from a ventilator (not represented in FIG. 1). The base body 2 further comprises an outlet 3b, which is intended to supply the breathing gas received from the ventilator to a patient. The base body 2 may further optionally comprise an attachment port 10 for a connector (not represented) to which a pressure measuring line (likewise not represented in FIG. 1) can be attached. The pressure measuring line is used to measure the pressure in the exhalation valve 1 for evaluation by the ventilator, if there is a need for this.

Located between the inlet 3a and the outlet 3b is an upper-side branch tube 4 with a free end 4a. The branch tube 4 is externally enclosed by a further cylindrical wall 5b of the base body 2, which forms an annular channel 5 with the branch tube 4. The wall 5b extends on the exhalation valve 1 only onto the regions of the branch tube 4 that are located inside the wall 5b. The annular channel 5 therefore extends between the branch tube 4 and the wall 5b only onto the upper region of the exhalation valve 1. The annular channel 5 does not extend to the opposite side, i.e. onto the lower region of the base body 2.

Provided in the wall 5b of the annular channel 5 is an outlet opening 6 which is formed by a multiplicity of openings in the wall 5b that concentrically enclose the branch tube 4. The opening 6 is formed by a multiplicity of elongate holes 6a, which are separated from one another by in each case a web 6b. The elongate holes 6a have rounded end regions. They are arranged distributed uniformly, i.e. symmetrically, along the circumference of the annular channel 5.

Located on the upper side of the free end 4a of the branch tube 4, or the free end of the wall 5b is a diaphragm 8 consisting of resilient material, in particular consisting of silicone, in order to ensure the valve function. The diaphragm 8 comprises a plane central part 8a, an outer edge region 8b and a curvedly running intermediate region 8c. In the assembled state of the exhalation valve 1, the outer region 8b is located in a downwardly oriented recess 7c of the cap 7.

In the closed state of the exhalation valve 1, the central region 8a of the diaphragm 8 lies on the free end 4a of the branch tube 4. The outer edge region 8b of the diaphragm 8 lies on the free end of the wall 5b. The diaphragm 8 is held by the cap 7, which is snap-fitted onto the upper end of the wall 5b. For this purpose, the wall has a preferably circumferential first latching recess 5d, which with a corresponding projection 7b on the cap 7 ensures a snap-fit or latching connection.

By virtue of the arrangement of the outlet opening 6, or the elongate holes 6a, in the immediate vicinity of the branch tube 4, breathing gas emerging from the branch tube 4 can leave the exhalation valve 1 via the annular channel 5 through the outlet opening 6 in an at least substantially laminar flow.

In order to individually adjust the outlet direction of the breathing gas leaving the outlet opening 6 according to requirement, a cover 9 of the outlet opening 6 is additionally provided, which is displaceable along the wall 5b, as a result of which, by displacing the cover 9, only the desired region of the outlet opening 6 is released and the other region thereof is covered.

According to FIG. 2, the cover 9 is configured for example as a partial ring bearing externally on the wall 5b and is provided, for example, with at least one inwardly directed projection 9a that engages in a corresponding external groove-shaped second latching recess 5c of the wall 5b. The arrangement may also be configured the other way round. In addition, the upper side of the partial ring engages in a recess formed by a step 7b on the cap 7 and the outer face of the wall 5b. In this way, the partial ring or the cover 9 is held securely, but at the same time the partial ring can be displaced along the wall 5b.

The cover 9 is located on the outer side of the wall 5b, bearing directly on the latter. In order to actuate the partial ring or the cover 9, elevations 11 or ribs may be formed on its outer side, and the cover 9 can be retained on these while being readily accessible with the surface of a finger and can be displaced according to requirements. The partial ring, or the cover 9, preferably covers an angular extent in the range of from 180° to 270°. The design according to the invention offers the advantage that the exhalation valve 1 according to the invention has an adjustable outlet opening 6, but is nevertheless distinguished by a simple design and can be assembled, i.e. mounted, easily.

FIG. 2 shows the exhalation valve 1 of FIG. 1 by way of example in the closed state, in which the diaphragm 8 lies with its central part 8a on the free end 4a of the branch tube 4 and is pressed by means of compressed air via the port 7a onto the free end 4a, in order to ensure a leak-free closure of the branch tube 4 when a control pressure is applied. In this position, breathing gas can flow from the breathing gas conditioning device through the exhalation valve 1 to the patient, without being able to escape through the branch tube 4.

When the patient exhales, the exhalation valve 1 needs to be opened. In order to open the exhalation valve 1, the compressed air supply at the port 7a is stopped so that, when a pressure is set up because of the exhalation activity of the patient, the central part 8a of the diaphragm 8 is lifted and the exhaled gas can escape first into the annular channel 5 and from there through the outlet opening 6 into the environment. The movement of the central part 8a of the diaphragm 8 is enabled here by the curvedly running intermediate region 8c of the diaphragm 8, which has only a very small layer thickness so that the central part 8a of the diaphragm 8 can be lifted even with a low pressure. In order to control the ventilator, the pressure may be measured via the attachment port 10, if such an option is provided in the ventilator. The ventilator provides the breathing gas according to a ventilation curve, optionally evaluates the available pressure measuring line and performs the valve control. This is carried out via the pressure measuring line (not represented), which is attached to the attachment port 10. For ventilators without this option, the attachment port 10 may be omitted from the exhalation valve 1 according to the invention without being replaced.

It may respectively be seen from FIG. 2 and FIG. 3 that the cap 7 can straightforwardly be snap-fitted by means of the projection 7b onto the wall 5b because of the first latching recess 5d there, and the diaphragm 8 can thereby be held securely in position.

As may likewise be seen from FIG. 2, one side of the annular channel 5 is obstructed by means of the cover 9 so that exhaled air can escape only through a partial region of the outlet opening 6. In FIG. 2, the closed region of the outlet opening 6 is the region that is assigned to the outlet 3b. On the other hand, the region of the outlet opening 6 that is assigned to the inlet 3a is open, as may also be seen from FIG. 2 and FIG. 3.

The present invention provides a simply producible exhalation valve having very favorable flow conditions in relation to the breathing gas exhaled by the patient, with simultaneously the possibility of varying the outlet direction of the breathing gas according to requirements.

### LIST OF REFERENCE SIGNS

1 exhalation valve

2 base body

3 passage channel

3a inlet

3b outlet

4 branch tube

4a free end of the branch tube

5 annular channel

5a open end of the annular channel

5b wall

5c second latching recess

5d first latching recess

6 outlet opening

6a elongate hole

6b web

7 cap

7a port for pressure line

7b projection on the cap

7c recess

8 diaphragm

8a central part of the diaphragm

8b outer edge region of the diaphragm

8c curvedly running intermediate region of the diaphragm

9 cover

9a projection

10 attachment port

11 elevation

1.-14. (canceled)

15. An exhalation valve, wherein the exhalation valve comprises:
a base body, the base body comprising:
a hollow passage channel with an inlet for receiving breathing gas from a ventilator and an outlet for supplying the breathing gas received from the ventilator to a patient,
a branch tube arranged between the inlet and the outlet and having a free end,
an annular channel externally delimited by a wall, enclosing the branch tube and likewise having a free end,
at least one outlet opening for breathing gas exhaled by the patient, returned again via the outlet and branched off via the branch tube,
a cap having a port for attaching a compressed air control line,
a diaphragm arranged at the free ends of the branch tube and the wall and, as a function of pressure applied on a side of the diaphragm averted from the open ends of the branch tube and of the annular channel, makes it possible for breathing gas exhaled by the patient and branched off via the branch tube to enter the annular channel,
the outlet opening being provided in the wall and being variable in respect of a flow direction of the breathing gas out of the outlet opening.

16. The exhalation valve of claim 15, wherein the annular channel encloses the branch tube concentrically.

17. The exhalation valve of claim 15, wherein the valve further comprises a cover which is variable in its position along a circumference of the annular channel.

18. The exhalation valve of claim 17, wherein the cover is displaceable with respect to the annular channel, or along the wall.

19. The exhalation valve of claim 17, wherein the cover 17 is guided by a step in the cap and/or a recess in the wall.

20. The exhalation valve of claim 19, wherein the cover is arranged on an outer side of the wall.

21. The exhalation valve of claim 17, wherein a rotary ring is provided as the cover.

22. The exhalation valve of claim 17, wherein the cover has an angular extent in the range of from 180° to 270°.

23. The exhalation valve of claim 15, wherein the outlet opening comprises a plurality of elongate holes distributed along a circumference of the annular channel.

24. The exhalation valve of claim 23, wherein the elongate holes are distributed uniformly along the circumference of the annular channel.

25. The exhalation valve of claim 23, wherein two adjacent elongate holes are separated from one another by a web.

26. The exhalation valve of claim 23, wherein the outlet opening comprises four elongate holes, which are arranged along the circumference.

27. The exhalation valve of claim 26, wherein elongate holes are arranged symmetrically along the circumference.

28. The exhalation valve of claim 15, wherein the annular channel or the wall extends at least substantially only onto a region of the branch tube.

29. The exhalation valve of claim 15, wherein the diaphragm closes the open end of the branch tube by compressed air being applied.

30. The exhalation valve of claim 15, wherein the diaphragm lies on an open end of the wall, and is fixed by the cap.

31. The exhalation valve of claim 15, wherein the cap comprises a recess for fixing the diaphragm.

32. The exhalation valve of claim 15, wherein the base body is present as a single piece.
